(19) United States
(12) Patent Application Publication
(10) Pub. No.: US 2002/0098493 A1
(43) Pub. Date: Jul. 25, 2002

(54) Drug-amino acids chimeric molecules

(76) Inventor: Asher Nathan, Palo Alto, CA (US)

Correspondence Address: James E. Eakin, McDermott, Will & Emerty, 3150 Porter Drive, Palo Alto, CA, 94304-1212, US

(21) Appl. No.: 09/865,966

(22) Filed: May 25, 2001

Related U.S. Application Data
(60) Provisional application No. 60/206,959, filed on May 25, 2000.
(60) Provisional application No. 60/207,369, filed on May 26, 2000.
(60) Provisional application No. 60/207,399, filed on May 30, 2000.
(60) Provisional application No. 60/232,615, filed on Sep. 14, 2000.
(60) Provisional application No. 60/259,231, filed on Jan. 3, 2001.

(51) Int. Cl. C12Q 1/68; C07H 21/04

(52) U.S. Cl. 435/6; 536/23.1

(57) ABSTRACT

The present invention relates to methods of using amino acid sequences for increasing the efficacy of existing drugs. In some embodiments, amino acid sequences are generated and combined with existing drugs to generate drug-amino acid sequence chimeric complexes that have superior pharmacological properties than the uncombined drugs. The chimeric complexes are designed to target only desired organs, without affecting other organs where the drug's activity is not desired, and to enhance drug bioavailability without losing the drugs' therapeutic properties.

### RELATED APPLICATIONS

This application is related to the following United States Provisional patent applications, each of which is incorporated herein by reference: U.S. Provisional No. 60/206,959, Filed: May 25, 2000, Title: Method For Identifying Genetic Diversity; U.S. Provisional No. 60/207,369, Filed: May 26, 2000, Title: Drug-Oligonucleotides Chimeric Molecules; U.S. Provisional No. 60/207,399, filed May 30, 2000, title: “Oligonucleotides with Pharmaceutical Properties; U.S. Provisional No. 60/232,615, filed Sep. 14, 2000, title: Synthetic Nucleic Acid Sequence Obtained by Molecular Evolution; and U.S. Provisional No. 60/259,231, filed Jan. 2, 2001, title: Drug-Oligonucleotides Chimeric Molecules; and to co-pending U.S. patent applications filed on even date herewith, the first of which is entitled “Drug-Oligonucleotides Chimeric Molecules”, S/N: ______, attorney docket no. 57557-012, and the second of which is entitled “Exta Cellular Drug-Oligonucleotides Chimeric Molecules”, S/N: ______, attorney docket no. 57557-014, both incorporated herein by reference.

### FIELD OF THE INVENTION

The present invention relates to methods of increasing the pharmacological activity of existing drugs, and more particularly relates to modifying known drugs by combining them with amino acid sequences to produce chimeric drug-peptide molecules having superior targeting, uptake and retention than the unmodified known drug.

### BACKGROUND OF THE INVENTION

Pharmaceutical and biotechnology companies currently select and optimize the majority of preclinical drug candidates based on their in vitro characteristics. Yet, in order for a drug to pass through all the regulatory hurdles needed to become an approved compound, it must also possess, by coincidence, the relevant in vivo characteristics. In vitro screens are based on the ability of each drug candidate to interact with a specific molecular target. The reason that most candidate drugs are not effective in the body is that their true pharmacokinetic properties cannot be adequately assessed in vitro.

Few lead compounds survive the preclinical and clinical trial processes. Each year an average pharmaceutical company will screen nearly 5,000 compounds as possible candidates for new medicines. Only 5%, or approximately 250 of the initially screened compounds, will survive to the preclinical laboratory testing stage. Out of the 250 compounds that successfully completed pre-clinical trial testing only five candidates, or 0.1% of the original 5,000 compounds, will pass the regulatory hurdles required to begin clinical trials in human subjects. Of the five potential therapeutics that any given company might have identified, only one of them will make it through to completion of phase III clinical trials and then be approved by the Food and Drug Administration (Source: IMS Health). These statistics highlight the gap between a drug's activity in a test tube and its pharmacological activity inside the body, and point to the inadequacies of in vitro selected drugs to successfully complete the preclinical and clinical developmental phases.

Many aspects of normal human physiology account for the reasons that successful in vitro lead compounds fail in vivo. The body prevents the drug from interacting with the target through a variety of mechanisms that involve absorption, distribution, metabolism, and excretion. For example, because most drugs travel to their target sites in a relatively nonspecific manner, sufficient quantities of the drug might never reach their intended sites in order to effect physiological change. Instead, drugs often become concentrated in healthy tissues and organs where they can cause damage. Although current drug discovery tools such as high-throughput screening (HTS) and rational drug design methodologies can select for improved in vitro activity, they are not generally useful for improving the drug's pharmacological activity inside the body. To make changes in a drug candidate's in vivo characteristics, generally require that medicinal chemists alter the drug's chemistry and then laboriously test each changed molecule, one at a time, in animal models. Due to the high cost and labor-intensive efforts needed to test each drug candidate in an individual animal, most drugs that fail during in vivo testing are discarded.

The inability to efficiently and accurately predict, let alone influence the outcome of a lead compound's behavior in vivo, negatively affects the time, cost, and level of risk associated with the drug development process. The average cost to develop a new drug, from the discovery phase through approval, is estimated to be $500 million dollars and, the process takes an average of ten to twelve years to complete. In addition to the high costs and long development times of drugs, the risk in drug development is extraordinarily high because the vast majority of preclinical candidates fail to become drugs. Even successful drugs that have gained regulatory approval generally have not been optimized for their in vivo characteristics and are thus prime candidates for improvement.

One factor that has increased the need for high-throughput in vivo optimization is the output from the Human Genome Project and the therapeutic drugs that it will generate. The Genome Project has created an explosion of potential targets, and by extension, the development of new drugs. In the history of drug discovery and development to date, all existing therapeutics has targeted fewer than 500 proteins, such as receptors, enzymes and ion channels. In contrast, it is now estimated that knowledge of the human genome will create an additional 10,000 to 60,000 new molecular targets, which should result in the development of many new drugs. Existing methods, including the newer versions of ultra high throughput screening (UHTS) are unlikely to efficiently screen the multitude of new lead candidates and yield approved drugs.

Typically attempts at improving existing drugs reside in chemically modifying the drug or, where the drug is the product of genetic techniques, modifying the sequence encoding the drug. Recent molecular techniques have made it feasible to simulate evolutionary processes and apply in vitro evolution to evolve molecules with novel properties that may have potential benefits for medical and industrial applications. In vitro evolution is a process of molecular discovery that mirrors the evolution of organisms in nature. In natural evolution, each organism contains a different DNA sequence, which is the genetic blueprint from which the organism is created. The DNA blueprint is continuously subjected to natural selection. Selection occurs through a process that has been described as survival of the fittest. Organisms that survive selection can pass on a portion of their DNA blueprint to their offspring. The offspring are themselves subjected to further rounds of selection and reproduction so that over time, there is an enrichment of the DNA sequences that impart improved survival qualities. In vitro evolution accelerates the process of natural selection.

To initiate the process of in vitro evolution in the laboratory, an initial population of randomly generated DNA molecules is synthesized. This random population is easily constructed using a conventional, commercially available nucleic acid synthesizer. The initial population of nucleic acid molecules is subjected to an artificial selection pressure whereby the molecules that have the desired behavior in vivo are retained and separated from the rest of the initial population, which is discarded. Rare DNA molecules that have the desired traits, as well as molecules that do not have the desired traits but due to the inaccuracy of the selection process have survived by chance, are amplified in vitro through application of an enzymatic process that exponentially amplifies DNA. Following amplification, the desired population is subjected to iterative rounds of selection and amplification, such that DNA molecules having the desired traits become enriched exponentially so that they may be identified using conventional screening techniques.(Tuerk, C., and Gold, L. 1990. Systematic evolution of ligands by exponential enrichment: RNA ligands to bacteriophage T4 DNA polymerase. Science 249 (4968):505-510.)

The first application of in vitro evolution was to evolve molecular diversity of mammalian antibodies (McAfferty, J. et al, (1990) Nature,348 (6301), 552-554; Kang, A. S. et al, (1991) Proc. Natl. Acad. Sci. USA, 88 (10), 4363-4366). This initially resulted in a vector system that produced F(ab) fragments of mammalian antibodies displayed as a VHCH1-pVIII fusion protein on the surface of M13 bacteriophage, or as a gene III-VCHC1 fusion protein in phage Fd (pVIII and the gene III protein are capsid proteins that are part of the exposed phage shell). These bacteriophage, when randomized, generate literally millions of antibody F(ab) fragments, and when the resultant cultures are immobilized they can be used as targets for hapten/antigen selection. The distinct advantage of this process is that very large combinatorial libraries of antibodies (Abs) can be expressed, of the order of 10⁵-10⁸ Ab sequence variations, which is far greater than by traditional hybridoma fusion methods. Phage display is based on this early work.

Phage display technology uses genes containing a spectrum of mutations directed to a target gene product (protein, antibody, or peptide) that are incorporated into a phage display vector containing a promoter and the gene III capsid protein of phage. This construct is used to transform E. coli cells (the phage host) resulting in a huge amplification of phage particles (typically 10¹⁰ or more). The phage express the gene III and target proteins as a fusion protein on its surface, where the protein is available for binding provided that the phage particle has been immobilized. The phage are then passed across a screen containing immobilized target molecules, and those that bind are eluted and sequenced. Molecular evolution is performed by iterating successive rounds of amplification, screening and selection, resulting in phage expressing mutants that bind their target receptor with nanomolar affinity or better. The process can be made more efficient by restricting the mutations suffered by the phage to codon-based mutations (i.e., every mutation results in the substitution of a sense codon.) The process is not limited to expressing antibodies however, proteins and peptides of all types are now routinely being randomized by “phage display” and screened against therapeutic targets. These phage expression systems lend themselves to efficient protein engineering by mutagenesis. Codon-based mutagenesis is also possible by randomly mutating the genes three bases at a time (ensuring that each mutation substitutes a unique amino acid). In this way mutagenesis, screening, and amplification of proteins again ligands (or of antibodies against haptens and antigens) can be accomplished extremely quickly (Maulik, S. and Patel, S. D. (1997) Molecular Biotechnology. Therapeutic Applications and Strategies. 83-88).

More recently, the application of in vitro evolution of nucleic acids in combination with the technique of phage display has been used to generate a variety of peptides which are screened in vivo to identify the translated sequences that specifically target certain organs (U.S. Pat. No. 5,622,699). Use of these techniques have led to the isolation of peptides that efficiently target molecules from blood to specific locations in the body, including brain, kidney, lung, skin and pancreas (Rajofte, D. et al (1998) J. Clin. Invest. 102 (2), 430-437; Pasqualini, R., and Ruoslahti, E. (1996) Nature, 280, 363-365). Peptides may have additional physiological properties conferred on them by their three-dimensional structure, by their charge, or by their capacity to interact with other nucleic or non-nucleic acid molecules. (Hermann, T., and Patel, D. J. Adaptive recognition by Nucleic Acid Aptamers. Science 287:820-825; U.S. Pat. No. 4,987,071) Small peptide moieties, called signal peptides, have also been identified and shown to mediate transport and targeting of large and diverse molecules between the nucleus and cytoplasm of cells, entry into subcellular compartments, secretion from cells, and uptake of molecules into cells from the surrounding fluid. In many cases, the relatively short signal peptide is sufficient to direct virtually any drug molecule to its destination. A striking example is a peptide derived from the Tat protein of HIV, which mediates very efficient entry of attached proteins into cells.

However, nucleic acid sequences are not only informational molecules, but may have additional physiological properties conferred on them by their three-dimensional structure, by their charge, or by their capacity to interact with other nucleic or non-nucleic acid molecules. (Hermann, T., and Patel, D. J. Adaptive recognition by Nucleic Acid Aptamers. Science 287:820-825; U.S. Pat. No. 4,987,071). Relatively short oligonucleotides posses structural diversity. Therefore, within a sufficiently comprehensive collection of such molecules, there will be members that can mimic the simple structures favored by nature for molecular addressing. Recognizing the ability of oligonucleotides to form a multitude of three-dimensional structures, Systematic Evolution of Ligands by Exponential Enrichment (SELEX™) was developed. SELEX™, is a combinatorial chemistry process that applies in vitro evolution to a very large pool of random sequence molecules to identify nucleic acid sequences and amino acid sequence that have the highest affinity for a variety of proteins and low molecular weight targets (Morris, K. N., Jense, K. B. Julin, C. M., Weil, M., and Gold, L. 1998. Proc. Natl. Acad. Sci. USA 96:2902-2907). The SELEX method is described in the following U.S. Pat. Nos.: 5,270,163, 5,475,096, 6,011,020, 5,637,459, 5,843,701 and 5,683,867, which are incorporated herein by reference. U.S. Pat. No. 6,011,020, discloses a method for producing chimeric molecules which comprise a nucleic acid region and a chemically reactive functional unit, wherein the nucleic acid region has a binding affinity for target, and the chemically reactive functional unit is a photoreactive group, an active-site directed compound, or a peptide. As in the other Gold patents the nucleic acid sequence is selected for its specific affinity for binding to a variety of molecular targets. Based on this criterion, the invention further relates to a method for targeting a therapeutic agent to a specific predetermined biological compuond. High affinity RNA ligands have also been identified (Homann, M, and Goringer, H. U. 1999. Nucleic Acids Res. 27(9):2006-2014.), and shown to bind to an invariant element on the surface of a living organism.

Another SELEX patent, U.S. Pat. No. 5,843,701, describes a method for producing high affinity polypeptide ligands that specifically bind to a desired target by reverse translation. Targets are screened using complexes comprising polypeptides that are expressed on the surface of phages and that are linked to the RNA that transcribes them. The advantage of this method is that since the nucleic acid that encodes the peptide remains associated with it, it is partitioned with the poplypeptide, with the means for further amplifying it by an in vitro process. As in the other SELEX patents, the ligand, which in this case is a polypeptide, is selected for its specific affinity for binding to a variety of molecular targets.

The success of the process of in vitro evolution has also been applied to evolve RNAs that contain cis-acting elements that are involved in nuclear transport, nuclear retention and inhibition of export of nuclear RNAs. In contrast to the nucleic acids of the SELEX patents, these RNA sequences were selected by their ability to localize in the nuclei of Xenopus oocytes (Grimm, C., Lund, E., and Dahlberg, J. E. 1997. Proc. Natl. Acad. Sci. USA 94:10122-10127; Grimm, C., Lund, E., and Dahlberg, J. E. 1997. EMBO J. 16:(4):793-?), and were not selected by their informational content nor by their ability to bind specific targets. This work indicates the ability of non-informational nucleic acids to affect their localization within cells.

A desirable approach to solving the abovementioned problems faced by the pharmaceutical industry would be to select drug candidates for clinical trials based on the in vivo pharmacological activity of the drugs. It would also be useful to modify several drugs simultaneously while selecting them under in vivo conditions. In addition it would be desirable to apply this approach to enhance the accessibility of known drugs to organs and tissues. Such an approach would yield a greater number of drugs that are effective in vivo in a timesaving and cost-efficient manner. Furthermore, it would be advantageous to exploit this knowledge in combination with exponential enrichment in vitro technology to identify amino acid sequences that when combined to known drugs, enhance the pharmacological activity of the known drugs.

### SUMMARY OF THE INVENTION

The present invention overcomes many of the limitations of the prior art by providing a new and novel method that exploits in vitro evolution in an in vivo setting for improving the pharmacological activity of known drugs. In general, the method of the present invention involves administering a library of phage-amino acid sequence-drug complexes to a mammal, isolating the complexes from at least one tissue of said mammal, identifying the nucleic acid encoding the amino acid sequence of the complex, amplifying the identified nucleic acid and combining it with known drugs to form chimeric amino acid-drug molecules that display a pharmacological activity superior to that of the unmodified drug.

An objective of the present invention is to provide a method that uses the methods of in vitro reverse translation or phage display to generate amino acid sequences to which known drugs are bound to generate an initial population of drug-amino acid sequence chimeric complexes that are subjected to iterative round of evolution to yiled an end population of chimeric complexes that are enriched in the species that increase pharmacological activity of the known drugs of the complexes.

Another objective of the present invention is to increase the relevance of the chimeric drug by first isolating a population of chimeric drugs using a first biological system such as cells in culture or cells isolated from organs of an animal, submitting said population of chimeric drugs to additional rounds of selection in cells from a second biological system such as cells derived from a human, and identifying the population of amino acid sequences that improves the accumulation of the chimeric drug within the human cells, so that the intracellular concentration of the chimeric drug is equal or greater than that attained in the cultured cells or those isolated from an organ of an animal. Thereafter, the desired chimeric molecules are used to increase tha pharmacological activity of known drugs in a patient.

Another objective of the present invention is to increase specificity of the selection. For example, if the chimeric drugs became concentrated in a desired organ such as the brain, as well as in an organ such as the kidney, where their accumulation is not desired, selection could be refined by including a negative selection step as follows: nucleic acids that encode the amino acid sequences of the chimeric drugs that localize in brain cells are amplified. The amino acid sequences encoded by the nucleic acids are combined with a known drug, and one half of the latter chimerc drugs are administered to an animal. Cells from the kidney are isolated and the nucleic acids encoding the amino acid sequences of the chimeric drugs that localize in the kidney are amplified. The nuceic acid sequences from the kidney cells are then used to perform subtractive hybridization with the nucleic acids sequences from the brain to yiled a population of nucleic acids that encode amino acid sequences that when combined with a known drug preferentially target the desired organ, which in this case is the brain.

Another objective of the present invention is to improve the bioavailabilty of drugs that are typically administered orally, whereby ingested chimeric drugs will be selected according to their ability to reach the circulation. Following oral administration of the initial pool of chimeric drugs, the nucleic acids encoding the Amino acid sequences of the chimeric drugs that reach the circulation are isolated from blood components, amplified, recombined with the drug and preferably, subjected to subsequent rounds of ingestion, isolation, amplification and combnation to yield an end population of chimeric drugs that are most likely to reach organ cells, thus increasing the bioavailability of drugs that are typically administered via the oral route.

It is another objective of the present invention to further increase the pharmacological activity of desired chimeric by mutagenizing the amino acid sequence encoding nucleic acid of the chimeric drug end population, combining the mutated nucleic acid with the known drug, and testing said mutated end population of chimeric drugs for desired properties that are enhanced over those of the non-mutated chimeric drug end population.

### DETAILED DESCRIPTION OF THE INVENTION

The present invention is based on the realization that it is possible to modify known drugs by binding an evolved amino acid sequence to the drugs to produce chimeric drug molecules that have pharmaceutical activities that are greater than those of unmodified drugs. The amino acid sequences may be derived from DNA or RNA, and are evolved by subjecting the amino acid sequence to one or more rounds of selection in vivo. The amino acid sequences of the present invention include peptides, polypeptides and proteins.

The present invention seeks to improve the pharmacological activity of known drugs, wherein pharmacological activity of a drug is defined as its ability to inhibit, agonize or antagonize a target by binding with the requisite affinity. Binding is achieved by a stereoelectronic interaction whereby the target of the drug recognizes the three-dimensional arrangement of functional groups and their electron and charge density. Prior biotechnology aims at developing ligands including nucleic acids, amino acids and small organic molecules to explore the three-dimensional “shape space” of their targets and bind to them with high affinity and specificity (Maulik, S. and Patel, S. D. (1997) Molecular Biotechnology. Therapeutic Applications and Strategies, 83-88). In contrast, the present invention aims at increasing the affinity and specificity of already known drug ligands by exploiting the potential three-dimensional arrangement of the drugs to increase the established desired properties of said drugs. In other words, the present invention does not select amino acid sequences for their specific binding properties.

In the context of this application the term therapeutic properties encompasses drug specificity, and bioavailability, which are directly related to the specific binding affinity of the drugs for a target. Drug specificity refers to the ability of a drug to target only the desired organ, without affecting other organs where the drug's activity is not desired, and drug bioavailability refers to the ability of a drug to reach a target site without losing its therapeutic properties.

The present invention seeks to improve these properties of known drugs by discovering amino acid sequences, preferably in vivo, that when combined with a known drug, increase the pharmaceutical activity of the drug by imparting increased specificty and bioavailabilty, as determined by an increase in the concentration of the chimeric oligonucleotide-drug molecules at the target organ, when compared to the intracellular concentration of unmodified known drugs.

The chimeric drugs of the present invention are expected to improve the therapeutic index, bioavailability, and the stability of the known drug, as well as the drug's known spectrum of activity, wherein therapeutic index relates to the ratio between the highest and lowest concentration known to have a desired pharmaceutical effect without causing harmful side-effects; stability of a drug includes the drug's resistance to enzymatic degradation, its clearance by the lymphatic and renal systems; and spectrum of activity refers to the drug's ability to simultaneously produce two or more beneficial effects. The properties of the drugs mentioned herein are merely examples, and any person versed in the art of pharmaceutics will be aware of the fact that other improvements in drugs may be desired.

The amino acid sequences of the chimeric drugs of the present invention may be derived from either RNA or DNA oligonucleotides that preferably have random sequences and that express the amino acid sequence in a ribosome display system as described in U.S. Pat. No. 5,843,701, or a phage display system as described in U.S. Pat. No. 5,622,699, respectively. Both US patents and the references therein are incorporated herein by reference in their entirety. DNA oligonucleotides are directly synthesized using a DNA synthesizer by methods known in the art. PCR is then used to synthesize the complementary second DNA strand to encode sequences containing the 32 possible amino acids. The double-stranded DNA is ligated in frame into a phage display vector, fuse 5, containing a promoter and the gene III capsid protein of the phage (U.S. Pat. No. 5,622,699). This construct is used to transform E. coli cells (the phage host) resulting in a huge amplification of phage particles (typically 10¹⁰ or more). The phage express the gene III and amino acid sequences as a fusion protein on its surface, where the amino acid sequences are available for combining with a known drug, to create a drug-amino acid sequence-phage complexes. The phage complexes are administered to a biological system, and at a predetermined time the phage complexes that have localized to a desired target, such as a specific type of cell, a tissue or an organ, are separated from the remainder of the phage complexes, and used to infect other host bacteria. Molecular evolution of the phage complexes is performed by iterating successive rounds of amplifying, combining, administering, and isolating, resulting in an end population of phage complexes that is enriched with informational nucleic acids encoding amino acid sequences that enhance the pharmacological activity of the known drugs of the phage complexes. The DNA encoding the amino acid sequences of the end population is subcloned into a sequencing vector to identify the desired sequence.

DNA oligonucleotides may be synthesized to contain the T7 promoter sequence so that they can be used to transcribe the population of RNA oligonucleotides using T7 polymerase, sequences that will transcribe into a eukaryotic ribosome binding site and a translation initiation codon, and a sequence encoding an amino acid sequence. The DNA is transcribed using T7 polymerase, and the resulting intial population of RNA oligonucleotides is translated in vitro. Translation is stopped prior to the release of the RNA and the encoded amino acid sequence from the ribosome, to yield an initial population of amino acid sequence-ribosome-RNA complexes (U.S. Pat. No. 5,843,701). Known drugs are combined with the initial ribosome complexes, and the resulting drug-ribosome complexes are administered to a biological system. At a predetermined time, the drug-ribosome complexes that have localized to a desired target, such as a specific type of cell, a tissue or an organ, are sequestered from the remainder of the drug-ribosome complexes. The RNA of the complex is separated from the drug-amino acid sequences and ribosome, it is reverse transcribed by RT-PCR, and translated in vitro to begin another round of evolution. In an alternative embodiment the ribosome is separated from the drug-amino acid sequence-ribosome-RNA complex, the amino acid sequence is covalently linked to the RNA, to form a drug-amino acid sequence-RNA complex, which is administered to the biological system.

Molecular evolution of the amino acid sequence of the ribosome complexes is performed by iterating successive rounds of amplifying, combining, administering, isolating and separating, resulting in an end population of ribosome complexes that is enriched with RNAs encoding amino acid sequences that enhance the pharmacological activity of the known drugs of the ribosome complexes. The pharmacological activity of the drug-amino acid sequence complexes is determined by the concentration of drug or of the amino acid sequences at the target site. Methods to determine free drug or drug-amino acid sequence concentration are described below. Finally, the RNAs of the end population are reverse transcribed, double stranded DNA is made using PCR and subcloned and sequenced in a sequencing vector, such as a PGEM vector.

The deduced amino acid sequence may be synthezised by methods known in the art, combined with known drugs, and used for therapeutic purposes.

In one preferred embodiment, an initial population of oligonucleotides is synthesized as described above. This initial population refers to a collection of between 10¹⁵ and 10¹⁸ oligonucleotides which differ from each other in sequence, and which encodes the amino acid sequences to yield an initial population of expressed amino acid sequences. Expressed amino acid sequences refers to the Amino acid sequences that are expressed by phage display or by the ribodome system described above.that are administered to a biological system at the beginning of the first round of evolution.

A biological system herein includes ex vivo and in vivo biological systems; wherein the ex vivo biological system comprises cells in culture that preferably are eukaryotic cell, or an isolated perfused organ, and the in vivo system comprises an animal or a patient. Further, the cell culture may comprise a single type or a plurality of diverse cell types; an isolated perfused organ may be for example an isolated perfused heart, or an isolated perfused kidney; an animal typically comprises smaller laboratory animals such as mice or rabbits, but may include larger species such as primates. The term cells includes prokaryotic and eukaryotic cells.

The term administering to a biological system refers to delivering in vivo a populations of phage-amino acid sequences or ribosome-amino acid sequences by any manner known in the art to administer pharmaceutical substances including oral, parenteral, rectal, nasal, topical administration, and may be formulated as a vaccine composition, together with any pharmaceutically or immunologically acceptable carrier, which is chosen in accordance with the preferred mode of administration. When the expressed amino acid sequences are administered to an ex vivo system, administering simply means adding the phage-displayed amino acid sequences or ribosome-associated amino acid sequences to the medium in which the cells are growing.

Following administration, expressed amino acid sequences, the complexes of interest, are isolated and the respective encoding nucleic acid sequences are identified as described below. The step of isolating cells targeted by the expressed amino acid-drugs complexes immediately follows the first step of administering said complexes to a biological test system. When the biological system is a cell culture, isolating means recovering cells that grow in suspension by known methods of centrifugation, or in the case where the cells grow adherent to the culture dish, isolating means detaching the cells either by trypsinization or EDTA, then collecting them by centrifugation. When the biological system is an animal, an organ or a tissue sample is obtained, the cells are dispersed by enzymatic means known in the art, and the cells are collected by centrifugation.

The expressed amino acid sequences are selected by their physical localization at the cell membrane of the target organ, and selection is not biased by the binding affinity the amino acid sequence may have for a three-dimensional target structure; the amino acid sequences are selected by their inability to bind to cells, and not by their specific binding to target molecules. A three-dimensional target structure herein refers to a structure to which an amino acid has been shown to bind specifically; wherein said target structures are those defined in any one of the SELEX™ patents. This selection criterion, in conjunction with the use of selection in vivo, fundamentally distinguishes the population of amino acid sequences obtained by the method of the present invention versus those that are identified in accordance with the method taught by the prior art including partitioning step required by certain of the prior art is not required in the present invention isolating the amino acid sequences that have entered the cell.

By using conventional oligosynthesis an enormous number of unique molecules, each embodying diverse structure and chemical topography can be created. Within these popualtions of amino acid sequences there will inevitably emerge substantial diversity of molecular structures. The population due to its large diversity, will contain members that will ferry drug payloads to targeted organs, possibly by mimicking a molecule that is naturally transported to the target organs.

In general, isolating the amino acid sequences means using methods that recover the nucleic acid sequences that encode the amino acid sequences portion of the drug-amino acid sequence complexes.

A subsequent population of amino acid sequence complexes refers to a population that is intermediate between the initial and end populations described above. The number of subsequent populations varies according to the number of iterative rounds of evolution that are required to reach a desired end population. An end population refers to a population of expressed amino acid complexes that is sufficiently enriched in one or more species of amino acid sequences that display desired properties. The desired properties of an expressed amino acid sequence include the inability to bind to a cell and the ability to target a specific organ. The ability to target a specific organ means that the amino acid sequence is able to “home in” to a target organ while avoiding another.

Ultimately, the end population of amino acid sequences is combined with a known drug to enhance the therapeutic properties of said drug. Combining a known drug to an a population of aminoacid sequences refers to a process whereby the known drug and the amino acid sequence are attached to each other by formation of covalent bonds.

Drug molecules used in the molecular evolution will also typically be fluorescent, immunologically or otherwise detectable, to allow detection of molecules in cells and tissue samples. Attaching drugs to peptides displayed on phages may be achieved by introducing surface-exposed cysteine residues into a phage protein by site-directed mutagenesis. The resulting sulfhydryl can be reacted in vitro with a maleimide or iodoacetamide derivative of the drug to create a stable covalent link. Alternatively, selection could be performed using a reporter molecule such as a fluorescent dye or green fluorescent protein (GFP) that is attached to the phages of the initial libraries. After the desired amino acid sequences are recovered, they will be made synthetically and attached to the drugs.

Separating a drug from an amino acid sequence after reaching the target can be achieved by incorporating cleavable bonds in the linker connecting the two moieties. Some examples include a disulfide bond that is susceptible to reductive cleavage in the that can be effected by reducing molecules such as glutathione, thioredoxine, or NADPH. The linker could incorporate a portion that is susceptible to hydrolysis by cellular enzymes. This could include short peptide-like chains recognised by proteases, such as cathepsins (in endosomes), calpains, caspases, proteasomes, subtillysin-like proteases, as well as extracellular proteases such as metalloproteases. Alternatively, cleavable esters, phosphodiester or other cleavable bonds can be incorporated and cleaved by the appropriate enzymes in vivo. Examination of the efficiency and localisation of the cleavage can be doneon test compounds in which a fluorophore is linked to the pepide in a way that its fluorescence is abolished; the fluorophore will become fluorescent only when properly released, allowing sensitive detection and measurement of the cleavage. Detection of specific peptides can be most reliably performed by immunoassays, using antibodies raised to the peptide. If a mixture of peptides is used, they must include a detectable moiety in common. This may be a short peptide that is an epitope for some existing antibody (“epitope tag”), or a non-peptide moiety that can be similarly detected.

The combining step produces a plurality of different chimeric molecule species, each specie being different from the other due to the sequence of its amino acid portion. Another diversity of the chimeric molecules may be denoted due to the fact that there will be times when the amino acid sequencesmay be bound to more than one position of the drug thus further increasing the possible combinations.

The word drug herein refers to any moiety, which may be an organic or inorganic molecule, a protein, peptide or polypeptide, a hormone, a fatty acid, a nucleotide, a polysaccharide, a plant extract, whether isolated or synthetically produced, etc. which is known to have a beneficial therapeutic activity, when administered to a subject, and includes drugs that are used to treat cardiovascular and neurological diseases, as well as cancer, diabetes, asthma, allergies, inflammation, infections and liver disease. The cardiovascular drugs include beta-blockers such as metoprolol and carvetilol, phosphodiesterase inhibitors such as milrinone, as well as other drugs including dobutamine and Angiotensin II antagonists. The neurological drugs include those drugs that by their combination with the amino acid sequences of the present invention, will be able to be shunted across the blood-brain barrier, as well as those drugs whose targeting of nerve cells will be improved by the amino acid sequences of the present invention. The cancer drugs include the taxanes such as Paclitaxel and Docetaxel, as well as Tamoxifen, gemcitabine, irinotecan, and cisplatin. In the case of diabetes, insulin is the drug whose efficiency is sought to be improved by the present invention. Asthma and anti-inflammatory drugs such as A1 adenosine receptor antagonists. Drugs to treat infection include antibiotics such as penicillin and cephalosporins, as well as new drugs that are currently used to treat M. tuberculosis infections.

There exist several standard methods for determing the concentration of a drug, and the methods should be tailored to each specific drug and its application. Typically, a cell extract is made from the tissue or organ, and the extract is clarified of proteins, and the resulting solution is subjected to a quantitative test. The circulating concentration of a drug or a chimeric drug is measured using clarified serum by the same methods used with cell extracts. Quantitative tests may be immunological, chromatographic, or be based on the binding properties of the drug to a target in vitro. Methods that can be used to measure the concentration of the extracellular chimeric drug as well as the free drug include Mass spectrometry, which is usually used in conjuction with liquid chromatography or high-pressure liquid chromatography (Font, E. et al (1999) 43 (12), 2964-2968; Kerns, E. H. et al (1998) Rapid Commun Mass Spectrom (England), 12(10), 620-624), and radio- and enzyme-linked immunoassays (Horton J K; et al (1999) Anal Biochem (United States), 271(1), 18-28; Goujon L; et al (1998) J Immunol Methods (Netherlands), 218(1-2),19-30). Methods that rely on the differences in the chemical properties of the chimeric and free drug can also be used. For example, since the chimeric molecule may be much larger than the free drug, gel filtration could be used. Otherwise, differences in partition between aqueous and various organic phases can be used as a method for separation. In some applications, the drug can be radioactively labelled, and the distribution of radioactivity can be followed. This method also allows to track the kinetic parameters of drug distribution, clearance and metabolism. Methods for determining the circulating concentration of a drug include radio- and enzyme immunoassays (Lelievre E; et al (1993) Cancer Res (United States), 53(15), 3536-40).

The probability of finding a drug molecule with optimal traits increases proportionately with the number of molecules that can be assayed. The invention allows for the simultaneous screening of up to 10¹⁸ amino acid sequence-encoding molecules. This level of molecules represents approximately a trillion-fold increase in screening capability than is possible using conventional high-throughput screens.

Alternate embodiments of the present invention include those described in the Summary of the Invention in the present application, as well as those given below as examples. However, having fully described a preferred embodiment of the invention, those skilled in the art will recognize, given the teachings herein, that equivalents exist which do not depart from the invention.

It may be appreciated by those skilled in the art that the literature describing the laboratory techniques needed to perform the processes of the present invention is extensive, and only exemplary references have been cited. All of the references cited herein are incorporated by reference in their entirety.

What is claimed is:

1. A method for improving the pharmacological activity of a drug, comprising:
a) creating an initial population of complexes of expressed amino acid sequences;
b) combining a drug with said complexes to produce an initial population of chimeric drug-amino acid complexes;
c) administering said drug-amino acid complexes to a biological system;
d) isolating said complexes associated with a target;
e) identifying nucleic acids encoding said expressed amino acid sequences;
f) amplifying the nucleic acids;
g) creating a subsequent population of expressed amino acid sequences;
h) combining said subsequent population with a drug to produce a subsequent population of drug-amino acid complexes;
i) repeating steps d through h at least twice to produce a final population of expressed amino acid sequences;
j) identifying the expressed amino acid sequences that increase the concentration of a drug to a target. and combining the amino acid sequence of the final population with a drug;

2. The oligonucleotides that encode the final population of expressed amino acid sequences of claim 1.
